(19) United States
(12) Patent Application Publication
(10) Pub. No.: US 2024/0252329 A1
(43) Pub. Date: Aug. 1, 2024

(54) METHOD AND SYSTEM FOR CONTROLLING PROSTHETIC DEVICE

(71) Applicant: Aether Biomedical sp.z o.o., Poznan (PL)

(72) Inventors: Dhruv Agrawal, Poznan (PL); Szymon Kwiatkowski, Wieniec-Zdrój (PL); Patryk Maczek, Kraków (PL)

(73) Assignee: Aether Biomedical sp. z o.o., Poznan (PL)

(21) Appl. No.: 18/696,389

(22) PCT Filed: Sep. 9, 2022
(86) PCT No.: PCT/IB2022/058506

§ 371 Date: Mar. 28, 2024

Related U.S. Application Data
(60) Provisional application No. 63/251,788, filed on Oct. 4, 2021.

(30) Foreign Application Priority Data
Oct. 4, 2021 (PL) 439137

(51) Int. Cl. A61F 2/72; A61F 2/50; A61F 2/58; A61F 2/68; G16H 10/60

(52) U.S. Cl. CPC A61F 2/72; A61F 2/586; G16H 10/60; A61F 2002/5036; A61F 2002/6827

(57) ABSTRACT

Disclosed is a method of controlling a prosthetic device. The method includes acquiring electromyographic (EMG) signals from one or more active electrodes configured to be in physical contact with a user, analyzing the acquired electromyographic (EMG) signals to determine the intent of the user and measuring one or more positional covariates associated with the user's residual limb. The method further includes controlling the prosthetic device in proportional response to the determined intent, wherein signal variations caused due to the positional covariates are compensated and providing multi-point sensory feedback to the user in response to the dynamics of the device, wherein the sensory feedback is provided via a wearable device that can be donned on or off by the user.

### TECHNICAL FIELD

The present disclosure relates generally to control of prosthesis; and specifically, to methods and system for controlling a prosthetic device.

### BACKGROUND

Prosthetics and prosthetic limbs have been used to replace human body parts since at least 1,000 B.C. Additionally, Egyptian, and Roman history is replete with recitations of wooden toes, iron hands and arms, wooden legs, feet, and the likes. However, it was not until the Renaissance that prosthetics began to provide for function (e.g., moving hands and feet) in addition to appearance. Prosthetic devices are generally worn by amputees on a missing or dysfunctional part of the body such as, arms, legs, joints, and the likes, to help the amputee in performing everyday activities with the assistance of the device. For example, an amputee having a missing leg may wear a prosthetic device on the missing leg.

Notably, prosthetic devices used in the past were purely mechanical and were limited to perform a few basic functions. In recent times, introduction of controllers in the prosthetic devices has helped in performing functions with the help of manual controls such as buttons or joysticks. However, such basic controllers do not take into consideration the dynamic conditions of the working environment and are limited to a small number of tasks. Moreover, another main challenge is that the user does not intuitively know the functioning of the prosthetic device.

Notably, existing solutions fail to provide proprioception to the user relating to the prosthetic device. For example, user has to constantly visually monitor the prosthetic device to be informed of its position. Additionally, highly evolved robotics hardware and software in the form of prostheses have been used to facilitate activities performed by a user. However, these methods are extremely extensive and may require costly surgeries.

Therefore, in light of the foregoing discussion, there exists a need to overcome the aforementioned drawbacks associated with existing solutions of controlling the prosthetic device.

### SUMMARY

The present disclosure seeks to provide a method of controlling a prosthetic device. The present disclosure also seeks to provide a system for controlling a prosthetic device. An aim of the present disclosure is to provide a solution that overcomes at least partially the problems encountered in prior art.

In one aspect, an embodiment of the present disclosure provides a method of controlling a prosthetic device comprising the steps of: acquiring electromyographic (EMG) signals from one or more active electrodes configured to be in physical contact with a user;
analyzing the acquired electromyographic (EMG) signals to determine the intent of the user;
measuring one or more positional covariates associated with the user's residual limb;
controlling the prosthetic device in proportional response to the determined intent, wherein signal variations caused due to the positional covariates are compensated; and
providing multi-point sensory feedback to the user in response to the dynamics of the device, wherein the sensory feedback is provided via a wearable device that can be donned on or off by the user.

In another aspect, an embodiment of the present disclosure provides a system for controlling a prosthetic device comprises:
one or more active electrodes configured to be in physical contact with a user to acquire electromyographic (EMG) signals;
a signal processing unit configured to analyze the acquired EMG signals to determine the intent of the user;
an inertial measurement unit configured to measure one or more positional covariates associated with the user's residual limb;
a controlling unit configured to control the prosthetic device in proportional response to the determined intent, wherein signal variations caused due to the positional covariates are compensated; and
a sensory feedback unit configured to provide multi-point sensory feedback to the user in response to the dynamics of the device, wherein the sensory feedback unit comprises a wearable device that can be donned on or off by the user.

Embodiments of the present disclosure substantially eliminate or at least partially address the aforementioned problems in the prior art, and enable intuitive controlling of the prosthetic device in a manner that improves proprioception of the user.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 illustrates a flowchart depicting steps of a method of controlling a prosthetic device, in accordance with an embodiment of the present disclosure;

FIG. 2 is a block diagram of a system for controlling the prosthetic device, in accordance with an embodiment of the present disclosure;

FIG. 3 is a schematic illustration of a system for controlling a prosthetic device, in accordance with an exemplary implementation of the present disclosure;

FIG. 4 is a flowchart listing steps involved in a process for training a machine learning model, in accordance with an embodiment of the present disclosure.

FIGS. 5A and 5B collectively illustrate steps involved in a process for controlling the prosthetic device, in accordance with an embodiment of the present disclosure;

FIG. 6 is a schematic illustration of a wearable device for providing multi-point sensory feedback to the user, in accordance with an embodiment of the present disclosure;

FIG. 7 is a flowchart listing steps involved in a process for providing multi-point sensory feedback to the user, in accordance with a specific embodiment of the present disclosure; and

FIG. 8 is exemplary schematic implementation of a wearable device for providing multi-point sensory feedback to the user, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a method of controlling a prosthetic device comprising the steps of:
acquiring electromyographic (EMG) signals from one or more active electrodes configured to be in physical contact with a user;
analyzing the acquired electromyographic (EMG) signals to determine the intent of the user;
measuring one or more positional covariates associated with the user's residual limb;
controlling the prosthetic device in proportional response to the determined intent, wherein signal variations caused due to the positional covariates are compensated; and
providing multi-point sensory feedback to the user in response to the dynamics of the device, wherein the sensory feedback is provided via a wearable device that can be donned on or off by the user.

In another aspect, an embodiment of the present disclosure provides a system for controlling a prosthetic device comprises:
one or more active electrodes configured to be in physical contact with a user to acquire electromyographic (EMG) signals;
a signal processing unit configured to analyze the acquired EMG signals to determine the intent of the user;
an inertial measurement unit configured to measure one or more positional covariates associated with the user's residual limb;
a controlling unit configured to control the prosthetic device in proportional response to the determined intent, wherein signal variations caused due to the positional covariates are compensated; and
a sensory feedback unit configured to provide multi-point sensory feedback to the user in response to the dynamics of the device, wherein the sensory feedback unit comprises a wearable device that can be donned on or off by the user.

The method and system of the present disclosure aims to provide an efficient, intuitive control of a prosthetic device. Notably, the present disclosure generates feedback in return of an action performed by the prosthetic device, thereby creating an interface between the prosthetic device and the patient. Furthermore, the present disclosure discloses a closed loop control system: sensing the user's intent, exerting proportional control, and providing sensory feedback. Moreover, the control system uses more than two channel controls which in turn makes the control more accurate. Notably, the system and method of the present disclosure capitalizes on the unique muscle synergistic patterns which are associated with intended hand movements. Additionally, the present disclosure is capable of simultaneously controlling multiple degrees of freedom (using hand and wrist simultaneously or individual finger control).

Throughout the present disclosure, the term “electromyographic (EMG) signals” refers to biomedical signals that measure electrical currents generated in muscles during their contraction representing neuromuscular activities. Notably, the electromyographic (EMG) signals are controlled by the nervous system and are dependent on the anatomical and physiological properties of muscles. Furthermore, the EMG signals are based upon action potentials at the muscle fiber membrane resulting from depolarization and repolarization. Additionally, EMG signals are herein used in prosthetic control as they represent electrical currents caused by the muscle contractions and actions. Beneficially, EMG signals are acceptable and convenient for the amputees as they can be acquired through surface sensors.

Throughout the present disclosure, the term “active electrodes” refers to non-invasive surface electrodes used for measurement and detection of EMG signals generated by a user. Additionally, the active electrodes assess muscle functions by recording muscle activity from the surface above the skin. Moreover, active electrodes record the muscle movements electrically from the muscle cells when they are electrically or neurologically activated. Beneficially, the one or more active electrodes amplify and digitize the EMG signals at a site of acquisitions, thereby providing better signal output in comparison with passive electrodes.

The method of controlling a prosthetic device comprises acquiring EMG signals from one or more active electrodes configured to be in physical contact with a user. Notably, EMG signals are recorded by placing the one or more active electrodes in physical contact to muscle groups of the user. Consequently, any movement in the muscle generates electrical signals that are captured by the one or more active electrodes. Furthermore, the one or more active electrodes are fabricated and fitted in an inner socket of a cuff attached to, for example, a limb of the user. Notably, the inner socket is the primary and critical interface between the amputee's residual limb and the prosthetic device. Additionally, the inner socket is structured in such a way that the one or more active electrodes comes in physical contact with the skin as soon as the prosthetic device is worn by the user. Furthermore, the inner socket ensures efficient fitting, adequate load transmission, stability, and control. In an example, there may be six to sixteen active electrodes depending on the user and the size of the inner socket.

Preferably, the method comprises filtering and amplifying the EMG signals, and digitizing the EMG signals into a format suitable for analyzing. Notably, the EMG signals are picked up by the one or more active electrodes. Additionally, the active electrodes have an analog front end that filters and amplifies the EMG signals to eliminate low-frequency or high-frequency noise, AC line noise, movement artifacts or other possible undesirable effects. Thereafter, the EMG signals are rectified and digitized into a suitable format for further analysis. Furthermore, the analog front end allows smaller footprint of the active electrodes, thereby allowing a larger number of electrodes that can be fitted in the cuff.

The method of controlling a prosthetic device comprises analyzing the acquired EMG signals to determine (detect) the intent of the user. The system for controlling a prosthetic device comprises a signal processing unit configured to analyze the acquired EMG signals to determine the intent of the user. Herein, a “signal processing unit” refers to an electronic unit that is capable of performing specific tasks associated with the aforementioned method and is intended to be broadly interpreted to include any electronic device that may be used for collecting, and processing EMG signals data from the one or more active electrodes. Moreover, the signal processing unit may include, but is not limited to, a processor, an on-board computer, a memory. The signal processing unit takes input from the one or more active electrodes and analyses the intent of the user using specific patterns of the EMG signals. Herein, the “intent” refers to the action that the user wants to perform using the prosthetic device. Moreover, the one or more active electrodes have a higher signal to noise ratio (SNR) which allows better accuracy in determining the intent of the user. Herein, signal to noise ratio (SNR) means higher signals and low noise. Consequently, signals with low noise help in determining the intent of the user more clearly.

Notably, the intent of the user may be to control the prosthetic device in order to perform one of a plurality of gestures by the prosthetic device. Additionally, the gestures may include, but are not limited to, movement of the prosthetic device, movement of one or more fingers of the prosthetic device, performing a specific gesture such as power grip, tripod grip, hook grip and the like. Moreover, the intent of the user may also determine the change in amount of force exerted by the prosthetic device. Furthermore, the force is determined based on the task performed by the prosthetic device. Herein, the signal processing unit analyses the EMG signals to identify specific patterns therein, wherein a given pattern in the EMG signal may be pre-associated with a given gesture of the prosthetic device. Upon identifying a specific pattern of the EMG signal, the signal processing unit is configured to determine the gesture associated with such pattern as the intent of the user.

In an embodiment, the method comprises
receiving training data related to the user, wherein the training data comprises EMG signal data corresponding to a plurality of gestures performed by the user during a training phase; and
providing the training data to a machine learning model for training thereof, wherein the machine learning model is configured to compute feature vectors for each of the plurality of gestures based on the EMG signal data.

Optionally, in this regard, an initial training is conducted in order to provide the machine learning model with training data of a given user. The training phase may be conducted at, for example, a prosthetic center. Notably, during the training phase, the user intends to perform a given gesture from a plurality of gestures one at a time, in multiple limb positions, and EMG signals generated corresponding to each of the plurality of gestures are recorded. The training data therefore comprises the EMG signal data and gestures corresponding thereto. Notably, the user may perform each gesture until enough data is collected. Additionally, the process is repeated for all the gestures required to be performed by the prosthetic device. Once sufficient data is acquired, the machine learning model is populated with the feature vectors generated using the training data set which customizes model for the individual user. Notably, the trained machine learning model may be allocated to or run by an external processor during the initial training process. Herein, the external processor may be connected to the machine learning model via a wired interface or a wireless interface.

Thereafter, the machine learning model computes feature vectors for each of the plurality of gestures based on the EMG signal data. Notably, EMG signal features are extracted in the form of time-domain (TD), frequency domain (FD) and time-frequency domain (TFD). Additionally, in the TD, the features are extracted from the variations of signal amplitude with time as per the muscular conditions. Moreover, the frequency domain uses the power spectrum density of the EMG signals for extraction of feature vectors. Furthermore, the combined features of time and frequency domain are used for time-frequency extraction (such as short Fourier transform and wavelets).

Optionally, the method further comprises classifying the EMG signals using a classification model to generate an intended gesture for the user. Optionally, the system comprises a grip controller configured to classify the EMG signals using a classification model to generate an intended gesture for the user. Herein, the machine learning model after training is employed as the classification model to determine the intended gesture of the user. Notably, the information gathered during feature extraction during the initial training stage is used to determine feature vectors corresponding to EMG signal data and generate intent of the user corresponding thereto. As mentioned previously, during the training stage, the user selects a particular gesture in order to generate the EMG signals with respect to the particular gesture, to be provided as training data and the machine learning model is populated with the feature vectors generated using the training data. Therefore, the classification model receives features from individual EMG sensors and generates feature vectors based thereupon. Once a feature vector is formed, it is compared with the feature vectors generated using the training data to determine the gesture intended by the user.

Preferably, the signal processing unit is disposed in a space between the residual limb and the prosthetic device, and is configured to communicate with the prosthetic device using a wired or wireless interface. Notably, the signal processing unit is placed inside the outer socket of the cuff attached to, for example, a limb of the user. Additionally, the signal processing unit is connected to the prosthetic hand using a wired interface or a wireless interface to pass on the detected gesture information. Additionally, the signal processing unit may house a battery and a Bluetooth® interface to connect the prosthetic hand and the signal processing unit.

The method of controlling a prosthetic device comprises measuring one or more positional covariates associated with the user's residual limb. The system for controlling a prosthetic device comprises an inertial measurement unit configured to measure one or more positional covariates associated with the user's residual limb. Herein, the term “inertial measurement unit” refers to an electronic device that decodes the position and orientation of the user's residual limb, using a combination of accelerometers, gyroscopes, and magnetometers. Notably, the inertial measurement unit may be situated in proximity to the signal processing unit and is responsible to calculate the positional covariates. The positional covariates associated with the user's residual limb include elbow angle, the angle between the axis of the forearm and the ground, hand height (relative to the user's shoulder) and the like. Notably, the positional covariates calculated by the inertial measurement unit helps in determining the position of the user's residual limb when performing a gesture or a task.

Optionally, training the machine learning model using training data relating to the positional covariates associated with the user's residual limb while the user performs the gestures in different residual limb positions. Notably, during the initial training, the positional covariates measured corresponding to each of the plurality of gestures performed in multiple limb positions, are also used as input data. Moreover, using positional covariates as training input data allows the machine learning model to be trained in such a way that it would not be affected by the different position of the limb during operation in real-life scenarios.

The method of controlling a prosthetic device comprises controlling the prosthetic device in proportional response to the determined intent, wherein signal variations caused due to the positional covariates are compensated. The system for controlling a prosthetic device comprises a controlling unit configured to control the prosthetic device in proportional response to the determined intent. Notably, the controlling unit uses the feature vectors, in real time, to generate the intended gesture and perform the intended gesture using the prosthetic device. Additionally, the controlling unit performs the gesture with the force intended by the user using the prosthetic device. Notably, the controlling unit further takes input from the inertial measurement unit to determine the positional covariates including elbow angle, hand height, and the likes as input vectors to be used during movement of the prosthetic hand. In an example, the prosthetic device is a prosthetic hand, and the controlling unit uses individually motorized fingers to manipulate the grip force and grip speed according to the user's intent. Additionally, the resultant action can either open or close a grip, or change the prosthetic hand to another position.

Optionally, in a case of a prosthetic hand, an electric motor and gear mechanism is housed together. Moreover, the gear has a pusher, to which a spring is attached that connects the pusher with the proximal part of the finger. Furthermore, the mechanism comprises a link connected to the distal part of the finger. Additionally, the inner side (the palm) of the prosthetic hand may be covered with a rubber gaiter. Notably, the electric motor and the gear mechanism helps in generating better gripping force when the user intends to close the fingers. Consequently, this mechanism helps the prosthetic hand grasp a heavier object more firmly and precisely.

Optionally, a user performing gestures generates multi-channel EMG signals. Additionally, the one or more active electrodes filter and amplify the EMG signals to eliminate low-frequency or high-frequency noise, or other possible artifacts. Thereafter, the signal processing unit computes feature vectors based on the features extracted from the EMG signal data. Furthermore, the signal processing unit, in communication with the grip controller employs the classification model to classify the EMG signal as at least one of: changing gesture of the prosthetic device, changing force exerted by the prosthetic device. Herein, if intent of the user corresponding to a given EMG signal is classified as the changing gesture of the prosthetic device, the classification model is operable to determine a confidence score for the determined intent. Thereafter, it is analyzed whether the gesture intended by determined intent of the user is an insignificant movement or an untrained gesture. Herein, an insignificant movement is EMG signal data with low signal values. In an event, the intended gesture is an insignificant movement or an untrained gesture, the dynamics of the prosthetic device are not changed. However, if the intended gesture is significant and known to the signal processing unit, the intended gesture is compared with the current dynamics of the prosthetic device. In an event the intended gesture is different than the current dynamics of the prosthetic device, the controlling unit controls the prosthetic device in accordance with the intended gesture. Alternatively, if the intent of the user corresponding to the given EMG signal is classified as changing force exerted by the prosthetic device, the controlling unit is configured control the prosthetic device to change the force exerted thereby.

Optionally, the method comprises receiving an input from the user in response to the generated gesture, in an event the generated gesture does not meet the intent of the user. Optionally, the system comprises an input means configured to receive the input from the user. Notably, the user may respond to a particular gesture on whether the intent of the user was rightly predicted or not. Additionally, the signal processing unit further has a calibration mode to recalibrate the trained machine learning model for all the gestures or some specific gestures, which might not be performing well. Additionally, the calibration mode functions similar to the training mode, but is not as extensive as the initial training. Notably, the input means may be an input device such as a controller or a mobile application executed on a mobile device.

Preferably, the method comprises providing the EMG signal data to the machine learning model for continuous training during routine usage of the device. Notably, during normal operation, the signal processing unit constantly saves the EMG signal data and the determined intent.

Moreover, this data is used to continuously train the machine learning model, for recalibration if required and to improve its accuracy.

Optionally, the system comprises a mobile, web or desktop application to support training, configuration, and maintenance of the device. Herein, the application refers to an application programming interface that provides the user with information relating to the system, and allows control, training and calibration of the signal processing unit. Additionally, such mobile or web applications may assist in remote training, configuration, and maintenance of the prosthetic device. Moreover, the user may provide an input using the application in an event the generated gesture does not meet the intent of the user. Beneficially, such input from the user ensures that data relating to misclassifications or errors is not provided to the machine learning model for training.

The method of controlling a prosthetic device comprises providing multi-point sensory feedback to the user in response to the dynamics of the device, wherein the sensory feedback is provided via a wearable device that can be donned on or off by the user. The system for controlling a prosthetic device comprises a sensory feedback unit configured to provide multi-point sensory feedback to the user. Notably, the sensory feedback unit may be a processing unit that upon receiving information relating to dynamics of the prosthetic device provides corresponding multi-point sensory feedback using the wearable device. Herein, the term “dynamics” refers to any change in position or force exerted by the prosthetic device. Additionally, the wearable device is an independent wearable device that may be connected using a wired or wireless connection with the sensory feedback unit. Moreover, the wearable device may be worn by the user on the residual limb or on other appendages. Furthermore, the sensory feedback unit takes movement and force information of the prosthetic device and provides feedback through the wearable device. Beneficially, the multi-point sensory feedback improves proprioception of the user and enables an intuitive management of activities performed using the prosthetic device. Furthermore, such feedback ensures that the user does not have to visually monitor the prosthetic device to identify movements thereof. Additionally, information such as force exerted by the prosthetic device, for example grip force of a prosthetic hand, cannot be effectively communicated by merely observing the prosthetic device. Therefore, such information can be efficiently communicated to the user using the multi-point sensory feedback.

Optionally, the multi-point sensory feedback is at least one of: a vibrotactile feedback, pressure feedback. Herein, the vibrotactile feedback refers to feedback provided via vibrations in the wearable device. The pressure feedback refers to feedback provided via pressure exerted by the wearable device.

Optionally, the wearable device is an autonomous band comprising one or more electromagnetic actuators configured to provide vibrotactile and/or pressure feedback to the user. Notably, the sensory feedback unit comprises electromagnetic actuators for providing the multi-sensory feedback to the user. Herein, the wearable device may comprise 4 to 16 electromagnetic actuators. Additionally, the electromagnetic actuators convey dynamic force and proprioceptive feedback to the user. Moreover, the electromagnetic actuators are all connected with each other through elastic elements. Furthermore, the elastic elements also act as a conduit for electrical connections between the electromagnetic actuators.

Optionally, the wearable device is connected to a motor-driven thread mechanism configured to pull a thread traversing the entire wearable device. Additionally, the wearable device houses a motor driven thread mechanism which traverses the whole wearable device. Moreover, the motor may contract and extend the wearable device by pulling on the thread or pushing on it. Notably, the electromagnetic actuators pressing against the arm sends vibrational feedback. Additionally, the wearable device contracts and expands against the arm using the motor mechanism and sends pressure feedback.

Optionally, a spatial mapping algorithm takes input from the prosthetic device on the action being performed, position of the fingers and the force being applied by the prosthetic device. Subsequently, the spatial mapping algorithm maps the action data to a specific stimulation pattern. Moreover, the spatial mapping algorithm generates output for the sensory feedback unit. In an example, the spatial mapping algorithm takes input for a grip as the amount of force exerted, the position of the hand. Thereafter, the spatial mapping algorithm maps the following data to a stimulation pattern and generates output.

Optionally, the multi point sensory feedback device is provided in a specific pattern to convey information on the dynamics of the prosthetic device to the user, wherein specific patterns are mapped to different dynamics of the prosthetic device and are calibrated to user's preference. Notably, each of the electromagnetic actuators is independent and capable of sending vibrational feedback with a different rhythm. Additionally, several combinations of the vibration feedback and the pressure feedback may be used in order to differentiate various dynamics of the prosthetic device. Moreover, the user may calibrate the feedback pattern best suited to them in accordance with specific dynamics or action.

Optionally, the prosthetic device is a prosthetic hand, and the wearable device provides dynamic patterns to the user in response to the dynamics of fingers of the prosthetic hand. Notably, the wearable device provides multi point sensory feedback to the user in response to the movement of fingers of the prosthetic device. Additionally, the user may calibrate different feedback patterns for each of the fingers in the prosthetic device. Moreover, depending on the feedback, the user gets a sense as to what action is being performed and the amount of force being exerted without having to actually look at it.

Optionally, the prosthetic device is a prosthetic hand, and the wearable device provides feedback of varying intensity in response to the grip force being applied by the prosthetic hand on an object. Notably, the wearable device is capable of providing feedback of different intensities in response to the grip force applied by the prosthetic hand. Additionally, the intensity of the feedback may increase when the grip force is high, and the intensity may decrease when the grip force is low. Consequently, grip force applied by the prosthetic hand to lift a heavier object would be higher and as a result the feedback intensity would be higher. Moreover, grip force applied by the prosthetic hand to lift a lighter object would be lower and as a result the feedback intensity would be lower.

Optionally, the sensory feedback unit receives finger position and grip strength from the prosthetic device. Additionally, fingers position and grip strength from the hand simulation are also received by the sensory feedback unit. Notably, the sensory feedback unit chooses the feedback pattern, calculates stimuli location, calculates stimuli frequency, and calculates the pulse-width modulation of the electromagnetic actuators. Notably, the sensory feedback unit sends the control signal to the one or more electromagnetic actuators. Subsequently, the one or more electromagnetic actuators send haptic stimulation via the one or more electromagnetic actuators to the user.

### DETAILED DESCRIPTION OF THE DRAWINGS

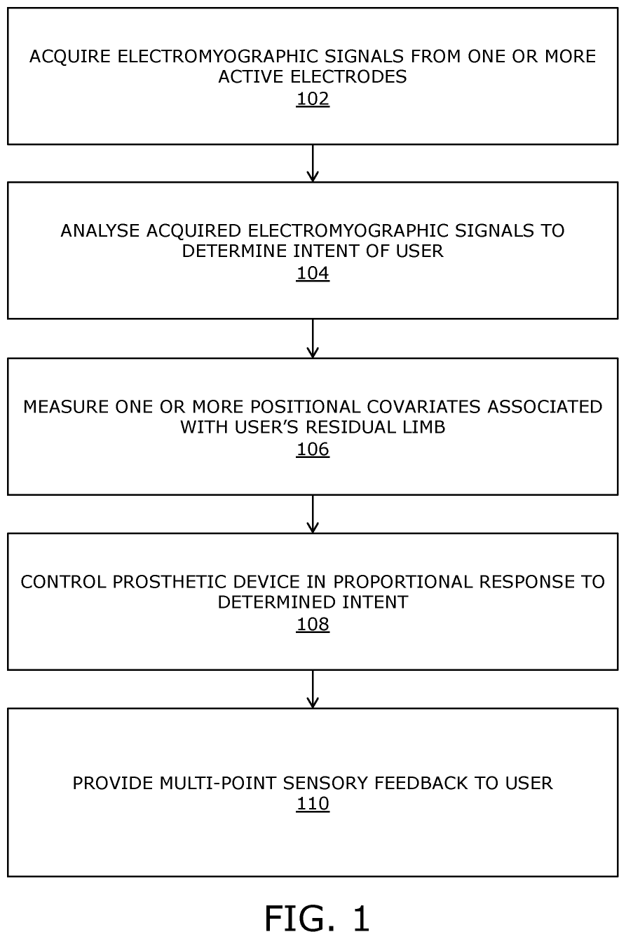

Referring to FIG. 1, illustrated is a flowchart depicting steps of a method of controlling a prosthetic device, in accordance with an embodiment of the present disclosure. At step 102, electromyographic (EMG) signals are acquired from one or more active electrodes configured to be in physical contact with a user. At step 104, the acquired electromyographic (EMG) signals are analysed to determine the intent of the user. At step 106, one or more positional covariates associated with the user's residual limb are measured. At step 108, the prosthetic device is controlled in proportional response to the determined intent, wherein signal variations caused due to the positional covariates are compensated. At step 110, multi-point sensory feedback is provided to the user in response to the dynamics of the device, wherein the sensory feedback is provided via a wearable device that can be donned on or off by the user.

The steps 102, 104, 106, 108 and 110 are only illustrative and other alternatives can also be provided where one or more steps are added, one or more steps are removed, or one or more steps are provided in a different sequence without departing from the scope of the claims herein.

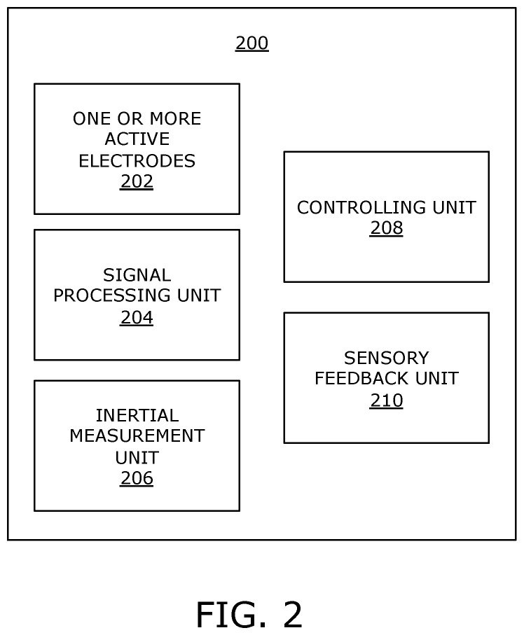

Referring to FIG. 2, illustrated is a block diagram of a system 200 for controlling a prosthetic device, in accordance with an embodiment of the present disclosure. The system 200 comprises: a one or more active electrodes 202, a signal processing unit 204, an inertial measurement unit 206, a controlling unit 208 and a sensory feedback unit 210. The one or more active electrodes 202 is configured to be in physical contact with the user to acquire EMG signals. The signal processing unit 204 is configured to analyze the acquired EMG signals to determine the intent of the user. The inertial measurement unit 206 is configured to measure one or more positional covariates associated with the user's residual limb. The controlling unit 208 is configured to control the prosthetic device in proportional response to the determined intent, wherein signal variations caused due to the positional covariates are compensated. The sensory feedback unit 210 is configured to provide multi-point sensory feedback to the user in response to the dynamics of the device, wherein the sensory feedback unit comprises a wearable device that can be donned on or off by the user.

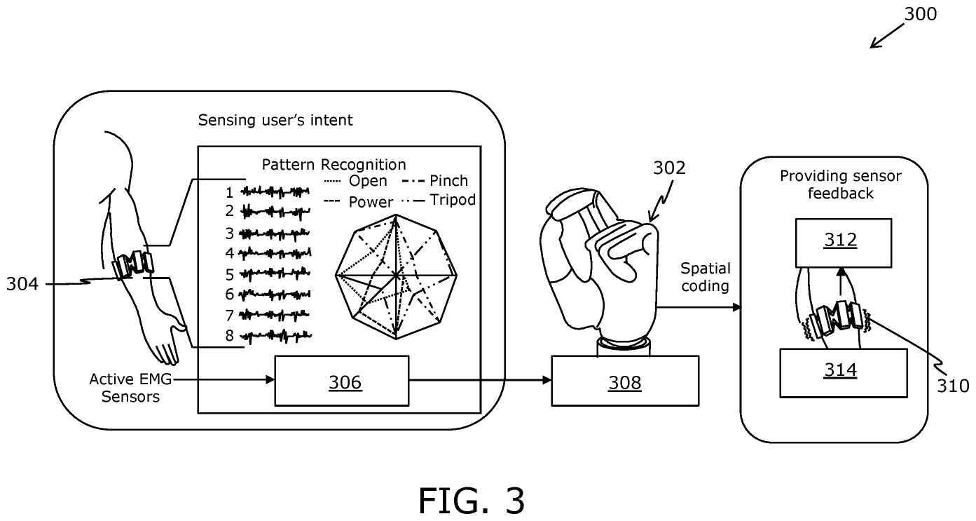

Referring to FIG. 3, there is shown a schematic illustration of a system 300 for controlling a prosthetic device 302, in accordance with an exemplary implementation of the present disclosure. Herein, the system 300 comprises one or more active electrodes 304 attached to an arm of a user. The prosthetic device 302 is worn on the residual limb of the user. Notably, the acquired EMG signals are provided to a signal processing unit 306 configured to analyse patterns in the EMG signals to determine intent of the user and extract feature vectors corresponding to determined intent of the user. The system comprises a controlling unit 308 configured to control the prosthetic device 302 in proportional response to the determined intent and in accordance with the feature vectors, wherein signal variations caused due to the positional covariates are compensated. Subsequently, a sensory feedback unit 310 is configured to provide multi-point sensory feedback to the user in response to the dynamics of the device. The sensory feedback unit 310 may provide vibrotactile feedback 312 and/or pressure feedback 314, thereby improving proprioception of the user.

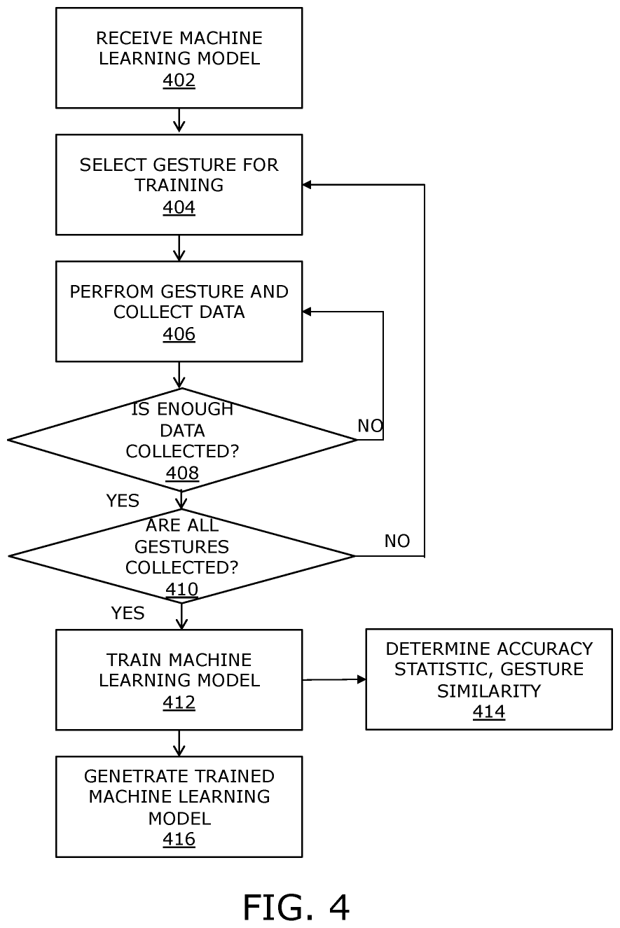

Referring to FIG. 4, illustrated is a flowchart listing steps involved in a process for training a machine learning model, in accordance with an embodiment of the present disclosure. The process includes, at step 402, receiving a machine learning model for training. At step 404, a gesture related to the user is selected for training. At step 406, the selected gesture is performed by the user and data is collected for forming a training data related to the selected gesture. At step 408, it is analysed if sufficient data relating to the selected gesture has been collected. If the collected data is insufficient, the process moves to the step 406. If the collected data is sufficient, the process continues to step 410, where it is checked whether data related to all gestures has been collected or not in the training data. If data related to all the gestures has not been collected, the process moves to step 404. If data related to all the gestures has been collected, the process continues to step 412, where the machine learning model is trained by providing the training data thereto. The process includes, at step 414, determining an accuracy, statistics and gesture similarity of the trained machine learning model. The process includes, at step 416, generating the trained machine learning model.

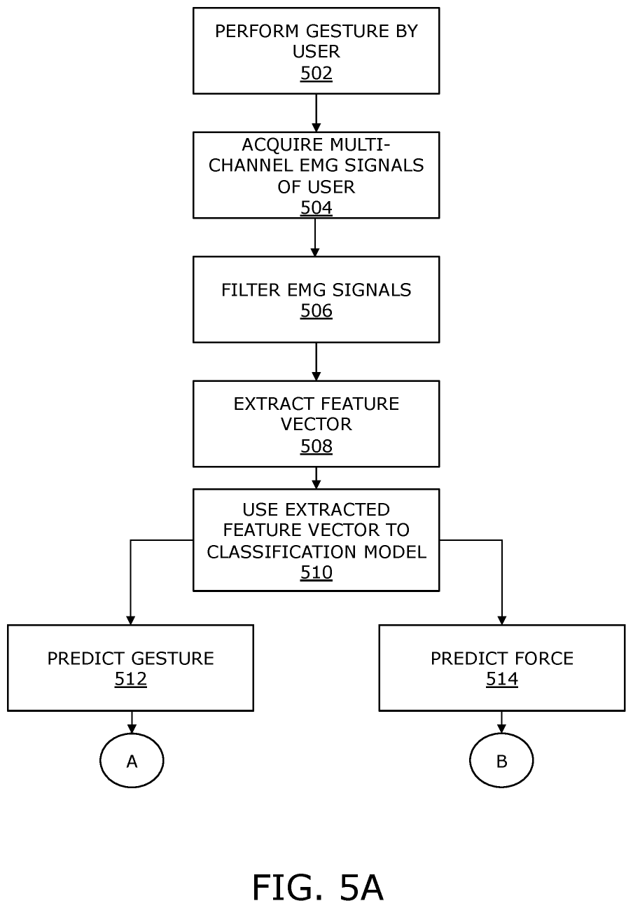

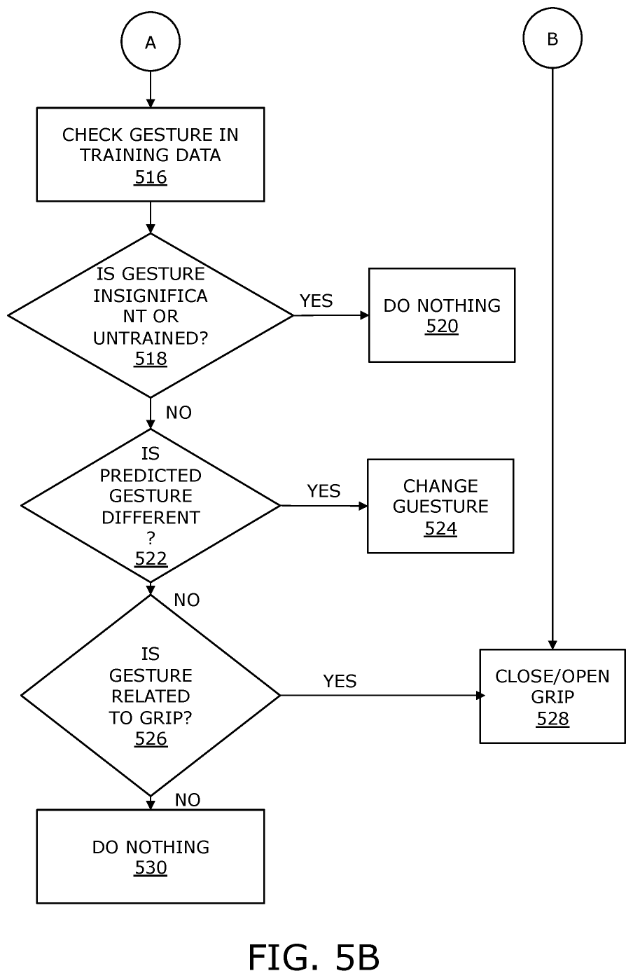

Referring to FIGS. 5A and 5B, illustrated collectively are steps involved in a process for controlling the prosthetic device, in accordance with an embodiment of the present disclosure. At step 502, gesture is performed by the user. The process includes, at step 504, acquiring EMG signals from one or more active electrodes configured to be in physical contact with the user when the user is performing the gesture. The process includes, at step 506, filtering the EMG signals. Herein, the EMG signals may also be amplified and digitized into a format suitable for analyzing. The process includes, at step 508, extracting feature vectors from the filtered EMG signal. The process includes, at step 510, providing the extracted feature vectors to a classification model, wherein the classification model is used to predict at least one of an intended gesture by the user at step 512 or an intended change in force exerted by the prosthetic device at step 514. At step 516, the predicted gesture is compared with the training data. At step 518, it is analyzed whether the gesture intended by determined intent of the user is an insignificant movement (low signal values) or an untrained gesture. At step 520, in an event, the intended gesture is an insignificant movement or an untrained gesture, the dynamics of the prosthetic device are not changed. However, if the intended gesture is significant and known, the intended gesture is compared with the current dynamics of the prosthetic device at step 522. In an event the intended gesture is different than the current dynamics of the prosthetic device, the controlling unit controls the prosthetic device in accordance with the intended gesture at step 524. If the intended gesture is not different than the current dynamics of the prosthetic device, at step 526 it is further analyzed whether the gesture relates to grip and a change in force thereof. If the gesture does relate to grip, corresponding change in grip is performed at step 528. Alternatively, if at step 514, the intent of the user corresponding to the given EMG signal is classified as changing force exerted by the prosthetic device, the prosthetic device is controlled to change the force exerted thereby at step 528. If at step 526, it is determined that the gesture does not relate to grip, no change in dynamics of the prosthetic device is made at step 530.

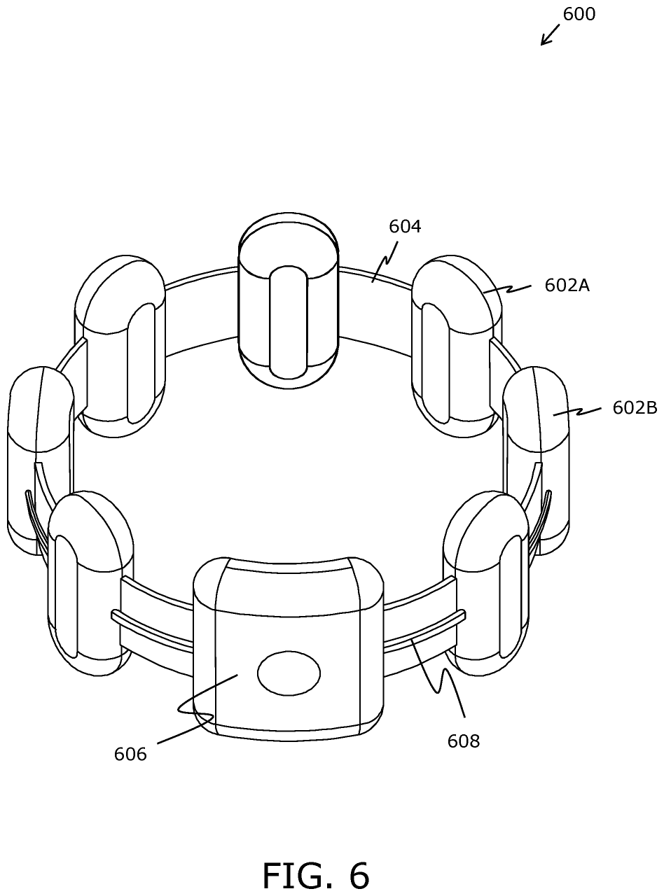

Referring to FIG. 6, there is shown a schematic illustration of a wearable device 600 for providing multi-point sensory feedback to the user, in accordance with an embodiment of the present disclosure. Herein, the wearable device 600 is shown in the form of a stretchable band that can be donned on or off by the user on a residual limb or any other part of the body. However, it is to be understand that any device of suitable shape and size can be used as a wearable device for this purpose. The wearable device 600 includes a plurality of electromagnetic actuators (such as, electromagnetic actuators 602A and 602B) for providing at least one of: a vibrotactile feedback, pressure feedback. The electromagnetic actuators 602A & 602B are connected via an elastic element 604 which also acts as a conduit for electrical connections between the electromagnetic actuators 602A & 602B and a main module 606. The main module 606 houses a on board computer to convey commands to the electromagnetic actuators 602A & 602B, for regulating vibration and force, in specific patterns. Optionally, the main module 606 contains a motor driven thread mechanism which pulls a thread 608 traversing the wearable device 600. The motor driven thread mechanism can contract and extend the wearable device 600 by pulling on the thread 608 and pushing on it.

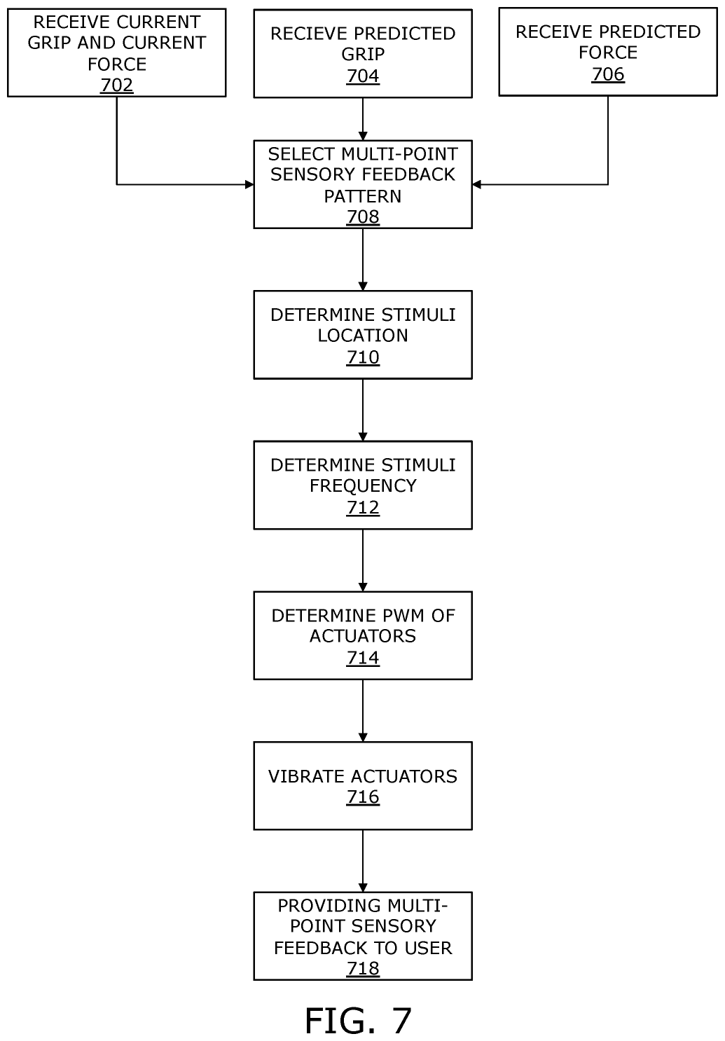

Referring to FIG. 7, illustrated is a flowchart listing steps involved in a process for providing multi-point sensory feedback to the user, in accordance with an embodiment of the present disclosure. The process, includes at step 702, receiving current grip or current force of grip being performed on the prosthetic device. Herein, the current grip may provide current position of fingers of the prosthetic device and the current force may provide current grip strength of the prosthetic device. The process, includes at step 704, receiving predicted grip. The process, includes at step 706, receiving predicted force. At step 708, a multi-point sensory feedback pattern is selected based on the current force and current grip strength, predicted grip and predicted force. At step 710, a stimuli location is determined. Herein, the stimuli location may define point or a combination of multiple points on the wearable device at which the multi-point sensory feedback may be provided to the user. The process, includes at step 712, determining a stimuli frequency. The stimuli frequency may be a frequency at which the electromagnetic actuators may vibrate. The process, includes at step 714, determining a pulse width modulation of electromagnetic actuators for providing a control signal. The process, includes at step 716, vibrating electromagnetic actuators according to the control signal. The process, includes at step 718, providing the multi-sensory feedback to the user by vibrating the electromagnetic actuators.

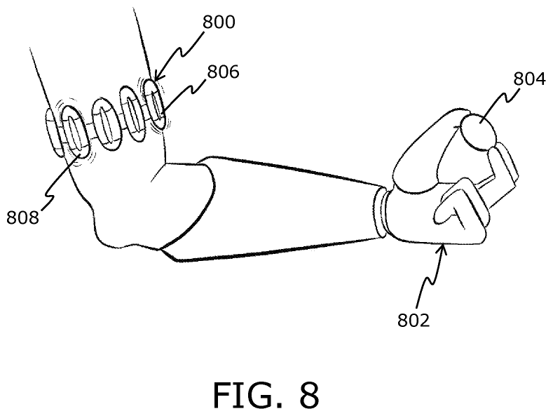

Referring to FIG. 8, illustrated is an exemplary schematic implementation of a wearable device 800 for providing multi-point sensory feedback to a user, in accordance with an embodiment of the present disclosure. In FIG. 8, a prosthetic hand 802 is holding an object 804, wherein the wearable device 800 is worn by the user on an arm. Herein, a plurality of electromagnetic actuators, such as electromagnetic actuators 806 and 808, are activated for providing the multi-point sensory feedback to the user. The combination of the plurality of electromagnetic actuators being activated with varying intensities indicates the amount of force applied by the prosthetic hand 802 on the object 804.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of controlling a prosthetic device comprising the steps of: acquiring electromyographic (EMG) signals from one or more active electrodes configured to be in physical contact with a user; analyzing the acquired electromyographic (EMG) signals to determine the intent of the user; measuring one or more positional covariates associated with the user's residual limb; controlling the prosthetic device in proportional response to the determined intent, wherein signal variations caused due to the positional covariates are compensated; and providing multi-point sensory feedback to the user in response to the dynamics of the device, wherein the sensory feedback is provided via a wearable device that can be donned on or off by the user.

2. The method of claim 1, further comprising: receiving training data related to the user, wherein the training data comprises electromyographic (EMG) signal data corresponding to a plurality of gestures performed by the user during a training phase; and providing the training data to a machine learning model for training thereof, wherein the machine learning model is configured to compute feature vectors for each of the plurality of gestures based on the EMG signal data.

3. The method of claim 2, further comprising training the machine learning model using training data relating to the positional covariates associated with the user's residual limb while the user performs the gestures in different residual limb positions.

4. The method of claim 2, further comprising providing the EMG signal data and determined gesture to the machine learning model for continuous training during routine usage of the device.

5. The method of claim 1, wherein the multi-point sensory feedback is at least one of: a vibrotactile feedback unit, pressure feedback unit.

6. The method of claim 5, wherein the multi point sensory feedback device is provided in a specific pattern to convey information on the dynamics of the prosthetic device to the user, wherein specific patterns are mapped to different dynamics of the prosthetic device and are calibrated to user's preference.

7. The method of claim 5, wherein the prosthetic device is a prosthetic hand, and the wearable device provides dynamic patterns to the user in response to the dynamics of fingers of the prosthetic hand.

8. The method of claim 5, wherein the prosthetic device is a prosthetic hand, and the wearable device provides feedback of varying intensity in response to the grip force being applied by the prosthetic hand on an object.

9. The method of claim 1, further comprising filtering and amplifying the EMG signals, and digitizing the EMG signals into a format suitable for analyzing.

10. The method of claim 1, further comprising classifying the EMG signals using a classification model to determine an intended gesture for the user and control the prosthetic device to perform the intended gesture.

11. The method of claim 10, further comprising receiving an input from the user in response to the generated gesture, in an event the generated gesture does not meet the intent of the user.

12. A system for controlling a prosthetic device comprises:
one or more active electrodes configured to be in physical contact with a user to acquire electromyographic (EMG) signals;
a signal processing unit configured to analyze the acquired EMG signals to determine the intent of the user;
an inertial measurement unit configured to measure one or more positional covariates associated with the user's residual limb;
a controlling unit configured to control the prosthetic device in proportional response to the determined intent, wherein signal variations caused due to the positional covariates are compensated; and
a sensory feedback unit configured to provide multi-point sensory feedback to the user in response to the dynamics of the device, wherein the sensory feedback unit comprises a wearable device that can be donned on or off by the user.

13. The system of claim 12, wherein the signal processing unit is disposed in a space between the residual limb and the prosthetic device, and is configured to communicate with the prosthetic device using a wired or wireless interface.

14. The system of claim 12, wherein the wearable device is an autonomous band comprising a plurality of electromagnetic actuators arranged along circumference of the autonomous band and configured to provide vibrotactile and/or pressure feedback to the user.

15. The system of claim 12, further comprises a grip controller configured to classify the EMG signals using a classification model to generate an intended gesture for the user.

16. The system of claim 15, further comprises an input means configured to receive an input from the user in response to the generated gesture, in an event the generated gesture does not meet the intent of the user.

17. The system of claim 12, further comprises a mobile, web or desktop application to support training, configuration, and maintenance of the device.
